(19) United States
(12) Patent Application Publication
(10) Pub. No.: US 2006/0142792 A1
(43) Pub. Date: Jun. 29, 2006

(54) Artery holder

(76) Inventor: Rafael Astudillo Ley, Lindingo (SE)

Correspondence Address: FAEGRE & BENSON LLP;PATENT DOCKETING, 2200 WELLS FARGO CENTER, MINNEAPOLIS, MN, 55402, US

(21) Appl. No.: 10/529,501

(22) PCT Filed: Sep. 17, 2003
(86) PCT No.: PCT/SE03/01451

§ 371 Date: Dec. 2, 2005

(30) Foreign Application Priority Data
Sep. 25, 2002 (SE) 0202817.3

(51) Int. Cl. A61B 17/08

(52) U.S. Cl. 606/157

(57) ABSTRACT

An artery holder for use in connection with heart surgery, which has a gripping and fixing member (1) having two co-operating first and second clamp elements (9, 10) which are arranged to grip muscle tissue (4) around an artery (5) in connection with its cut end (7) and clamp muscle tissue while simultaneously fixing the artery and exposing its cut end. Furthermore, the artery holder has a spring member (2) to actuate the gripping and fixing member with a predetermiined spring force in order to clamp the muscle tissue, and an actuating member (3) to actuate the gripping and fixing member against the action of the spring force from the spring member in order to open the clamp elements for receiving muscle tissue therebetween and subsequent release of clamped muscle tissue.

The present invention relates to an artery holder for use in connection with heart surgery.

When performing coronary vessel surgery by means of bypass operation the blood is conveyed past the narrow places in the coronary vessels which are the reason for an impaired heart function. New vessels are operated therein, said new vessels being intended to increase the blood flow to the myocardium by conveying the blood past the narrow places in the coronary vessels. For instance, the new vessels may be formed from some of the mammaria arteries, usually the left one, or both. The mammaria arteries are arteries extending right beside and in parallel with the sternum on the inside of the thorax wall. When the left mammaria artery is utilised, it is connected to the anterior descending branch of the left coronary artery. When performing the operation the mammaria artery is made available from the inside of the thorax wall and is cut in order to form a free end.

Simultaneously, an opening is performed in the coronary vessel in a position located downstream the stenosis, to which opening said free end of the mammaria artery is to be connected by stitching by means of a suture.

The mammaria arteries are enclosed by muscle tissue. When performing operation forceps or lockable instruments provided with jaws are utilised in order to hold the end portion of the mammaria artery and fix its end in a correct position in relation to the opening of the coronary artery. The holding and fixing is done by means of the forceps or another instrument clamping a portion of the muscle tissue around the free end portion of the mammaria artery. By means of a forceps, or several forceps or another instrument, however, it is difficult to expose the free end of the mammaria artery in a satisfactory manner in order to make it entirely visible and available for stitching to the coronary artery. Furthermore, there is a risk of damaging the inner surface of the mammaria artery by the needle opposite each place where the needle is stuck through the artery wall. Sometimes, a forceps is also used to be brought into engagement with the artery wall itself, implying that the portion of the forceps which is inserted into the artery easily can damage the inner surface of the artery wall. It is also known to utilise other types of instruments for holding the mammaria artery, said instruments being inserted into the mammaria artery. Also in this case there is a risk of damaging the inner surface of the artery wall. Damages on the inner surface of the artery wall, being caused by the stitching needle or a holding instrument, could later increase the risk of the emergence of clots of blood. The forceps are operated manually, and therefore the clamping force on the clamp elements can be difficult to control, implying that clamping force which is too large may be applied, which could damage the muscle tissue, or that a clamping force which is too small may be applied, implying that the forceps looses its grip on the muscle tissue. Furthermore, it is difficult to hold a forceps in one hand without movement of the forceps, resulting in a change of position of the artery end in relation to the opening in the coronary vessel. With respect to the jaw-equipped instruments, the jaws are locked in their operative clamping position by means of a locking device. Also in such cases, the clamping force may become too large, so that the muscle tissue is damaged.

The object of present invention is to provide an improved artery holder for heart surgery which at least substantially reduces the above-discussed problems.

The artery holder according to the invention is characterised in that it comprises
a gripping and fixing member including two mutually interacting first and second clamp elements being arranged for gripping muscle tissue, that encloses an artery, in connection with a cut end of the artery, and for clamping muscle tissue while simultaneously fixing the artery and exposing said cut end,
a spring member being arranged to actuate the gripping and fixing member with a predetermined spring force in order to achieve said clamping of the muscle tissue, and
an actuating member being arranged to actuate the gripping and fixing member against the action of the spring force from the spring member in order to open the clamp elements for enabling reception of muscle tissue therebetween and subsequent release of clamped muscle tissue.

The invention will be described further with reference to the drawings.

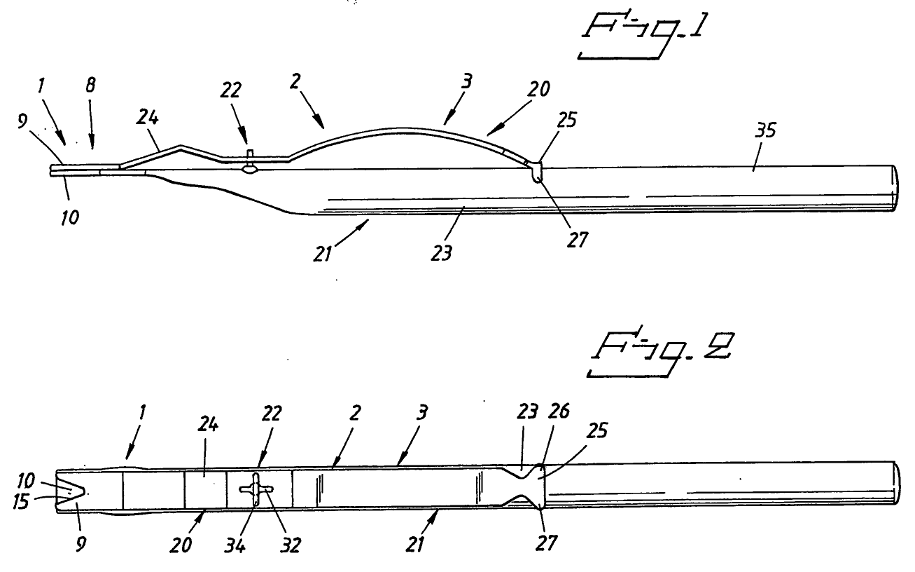

FIG. 1 is a lateral view of an artery holder according to the invention including first and second structural elements.

FIG. 2 is top view of the artery holder according to FIG. 1.

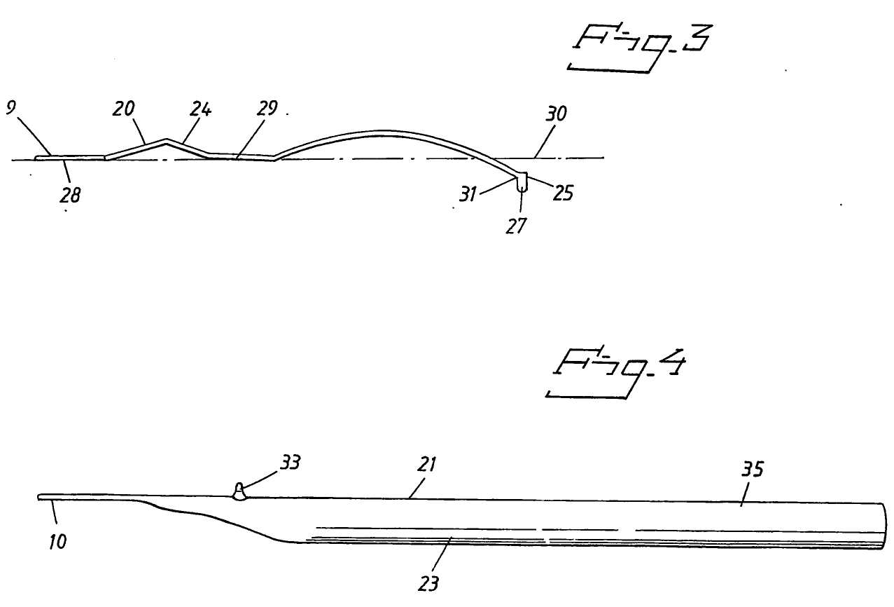

FIG. 3 is a lateral view of the first structural element of the artery holder.

FIG. 4 is a lateral view of the second structural element of the artery holder.

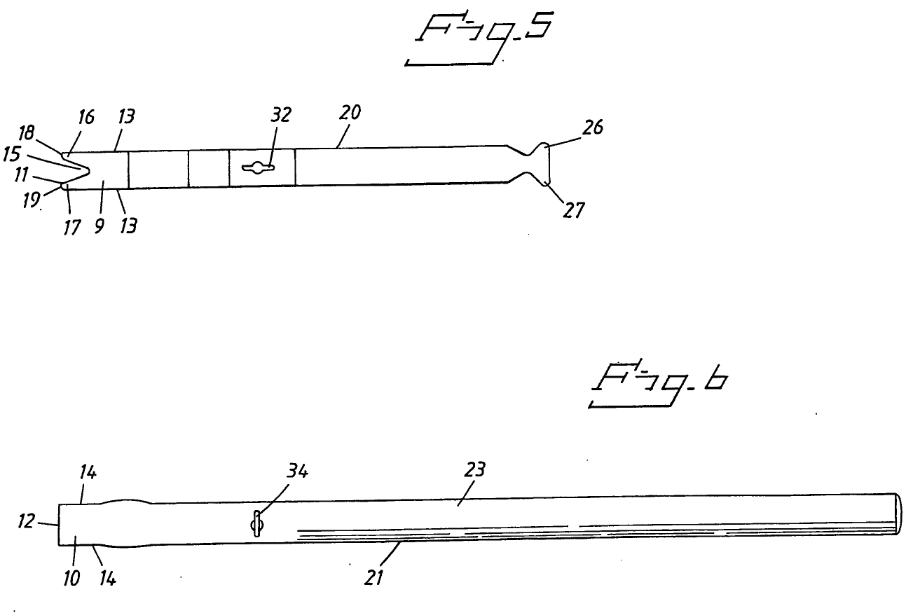

FIG. 5 is a top view of the first structural element.

FIG. 6 is a top view of the second structural element.

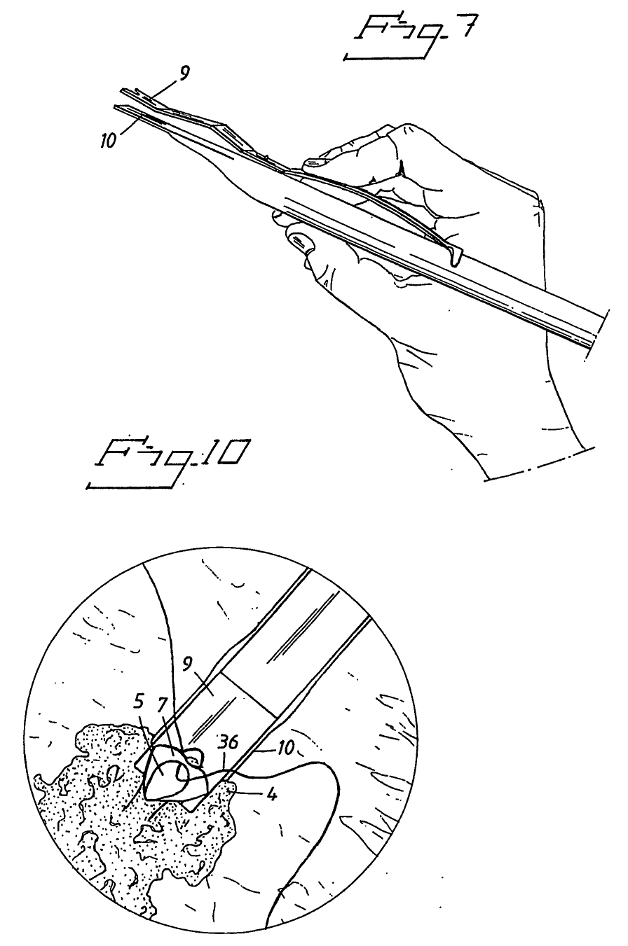

FIG. 7 is a perspective view of the artery holder according to FIG. 1 in an opened condition by means of a force applied by a hand gripping the artery holder in the same fashion as a pen.

FIG. 8 shows the artery holder in an opened condition while being applied against a cut artery.

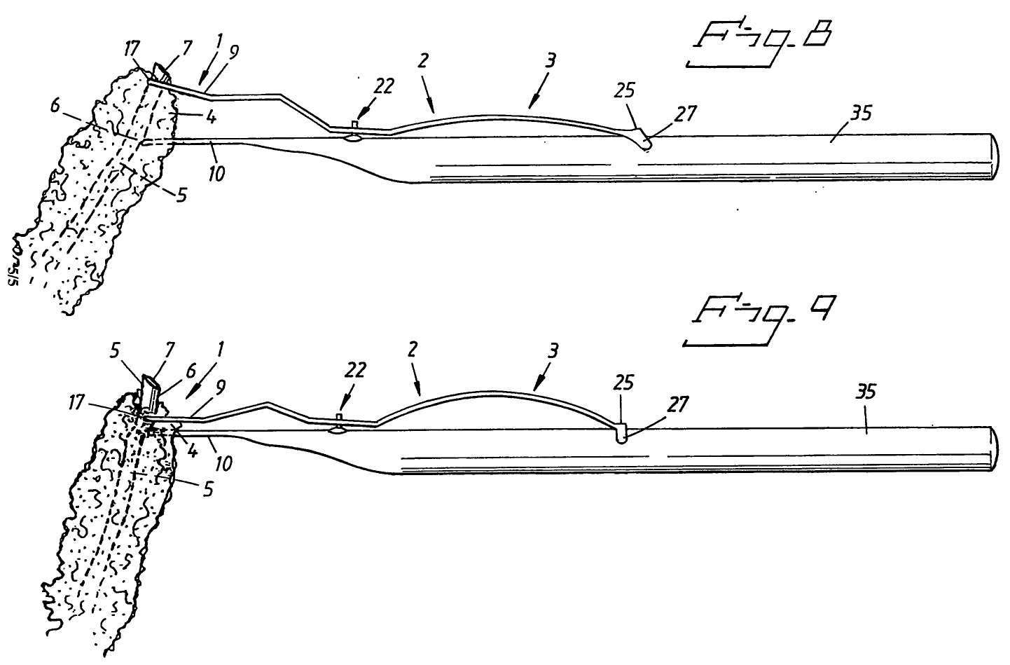

FIG. 9 shows the artery holder according to FIG. 8 in an operative position while fixing the artery.

FIG. 10 is an enlarged portion of the artery holder while fixing the artery, seen substantially from above in FIG. 9.

In the drawings, an artery holder is shown for use in connection with heart surgery, and more particularly coronary vessel surgery. The artery holder has a straight elongated shape, and includes a gripping and fixing member 1, a spring member 2, and an actuating member 3. The gripping and fixing member 1 is arranged for gripping a portion of the muscle tissue 4 (see FIGS. 8, 9 and 10), enclosing the artery 5 and being present in connection with an end portion 6 of the artery, and for clamping the muscle tissue 4 while simultaneously fixing the artery 5 and exposing its end portion 6 which terminates in an end 7. For this purpose, the gripping and fixing member 1 includes first and second clamp elements 9, 10 being movable in relation to each other in order to be opened and closed. In the closed position, the clamp elements 9, 10 are acting against each other with a predetermined spring force in order to clamp muscle tissue and fix the artery 5 in a firm grip. In the opened position, the distance between the clamp elements 9, 10 is sufficiently large so that muscle tissue 4 can be received in the space which is created in this way (see FIG. 8). The clamp elements 9, 10 have planar surfaces facing each other, and front end edges 11, 12 of different shape, extending between the parallel lateral edges 13 and 14, respectively, of each clamp element 9, 10. The front end edge 12 of the second clamp element 10 is straight and forms a right angle with the lateral edges 14, whereas the end edge 11 of the first clamp element 9 is retracted in order to form a central recess 15 having a predetermined shape and length, so that at least a portion of the artery 5 can be received in the recess 15 when the artery holder is brought into an operative engagement position with an inclined alignment in relation to the artery 5. The central recess 15 and each lateral edge define therebetween a tongue 16 and 17, respectively. The tongues 16, 17 constitute advantageous lateral supports for the artery, so that the artery is fixed in a lateral direction. The tongues 16, 17 are similar in shape, and their outer, free ends 18, 19 are positioned in line with or almost in line with the transversal end edge 12 of the second clamp element 10. The recess 15 suitably has a V-shape, thereby enabling arteries of different cross-sectional dimensions to be received in the recess 15.

The above-mentioned spring member 2 is arranged to actuate the gripping and fixing member 1 with said predetermined spring force in order to achieve said clamping of the muscle tissue 4 between the two clamp elements 9, 10.

The above-mentioned actuating member 3 is arranged to actuate the gripping and fixing member 1 against the action of the spring member 2 in order to open the gripping and fixing member 1 for enabling reception of muscle tissue 4 and subsequent release of clamped muscle tissue 4 when the artery has been stitched to the coronary vessel by means of a suture 36. FIG. 10 shows the suture 36 brought through the artery in an initial step, in order to subsequently be brought through the coronary vessel (not shown).

The artery holder can be designed in many different ways in order to obtain a construction including the three functional main components denoted 1, 2 and 3 and described above. In the Figures, an embodiment of the artery holder is shown which, at present, is preferred, and which includes first and second structural elements 20, 21 which together form the gripping and fixing member 1. The structural elements 20, 21, thus including each one of said first and second clamp elements 9, 10, are detachably connected to each other by means of an attachment member 22 being arranged at a predetermined distance from the free end edge 11 of the first clamp element 9 in order to form a turning point for the first clamp element 9.

In addition to the second clamp element 10, the second structural element 21 includes a straight, round supporting body 23, such as a tube or a massive rod, from which the blade-shaped clamp element 10 projects linearly from the supporting body 23. The second clamp element 10 and the supporting body 23 preferably are designed integral with each other.

The above-mentioned actuating member 3 constitutes a portion of the first structural element 20, viz. the portion extending from the attachment member 22 in a direction backwards from the gripping and fixing member 1. Between the first clamp element 9 and the attachment member 22, the first structural element 20 has a raised, arc-shaped or angular section 24, whereby the first clamp element 9 obtains a distinct contact against the second clamp element 10. The actuating member 3 has an arc-shape, and its rear end portion is designed as a movable support 25 having two arc-shaped lateral legs 26, 27 for straddling the round supporting body 23 of the second structural element 21. The support 25 is in sliding contact with the supporting body 23 and is guided along this forwards and rearwards, when the actuating member 3 is subjected to external and internal forces 20 directed transversely towards and away from, respectively, the second structural element 21.

The above-mentioned spring member 2 is formed by the arc-shaped actuating member 3. To this end, the first structural element 20 consists of a resilient material, preferably a spring steel, and is designed as a spring plate having been curved and bent in a predetermined fashion. By means of the attachment member 22 and the support 25, the arc-shaped actuating member 3 is spring-loaded against the second structural element 21, so that a spring force is transferred to the gripping and fixing member 1, the two clamp elements 9, 10 of which thereby acting against each other with a predetermined clamping force being sufficient for clamping muscle tissue 4 and fixing the artery 5. In order to achieve this spring-loading, the spring plate 20 is pre-bent and pre-curved so that it, in an unaffected condition, has the two points of contact 28, 29 being represented by the first clamp element 9 and the location of the attachment member 22, located in a straight line 30 intersecting the arc-shaped actuating member 3 above the support 25 which subsequently forms the third point of contact 31 when spring-loading. The positions of the three points of contact 28, 29, 31 in relation to each other are determined and obtained by means of suitable curving and bending of a flat blank, so that the predetermined clamping force is achieved between the two clamp elements 9, 10.

In the embodiment shown, the attachment member 22 is formed partly by a longitudinal, narrow aperture 32 in the first structural element 20, and partly by a T-shaped engagement body 33 (see FIG. 4) being anchored to the second structural element 21, and the horizontal engagement portion 34 of which is extending transversely to said second structural element. Thereby, the two structural elements 20, 21 are easy to put together in order to form the finished artery holder, and easy to disassemble after use for sterilisation. When performing the assembly, the two structural elements 20, 21 are positioned so that they cross each other perpendicularly, after which the aperture 32 is aligned in relation to the engagement portion 34 so that this can be brought through the aperture 32. By means of turning the spring plate can be brought into a retained, spring-loaded position on the supporting body 23 and its clamp elements 10 (se FIGS. 1 and 2). The disassembly is carried out in a reverse order, after having lifted up the support 25 a small distance from the supporting body 23.

The supporting body 23 is extended backwards in order to form a handle 35 for reliable holding of the artery holder in one hand.

In another embodiment (not shown), the artery holder also is made from two structural elements, but the structural elements are united, for example within the region of the actuating member 3, and together they form an upper portion and a lower portion of the artery holder. Both structural elements consist of spring plates, being curved and bent in order to form the three main members being denoted 1, 2 and 3 above, wherein the transition between the two portions has the shape of an arc forming a handle for being held by one hand. Within the upper portion of the artery holder, the arc merges into the actuating member which, also in this case, acts like a spring member. The two portions are fixed to each other by means of an attachment member, e.g. in the form of a rivet, in order form said gripping and fixing member.

The artery holder shown is intended for use in open heart surgery. It can, of course, also be utilised for endoscopic surgery by means of being provided with a suitable manipulating mechanism (not shown) for external, manual operation of the artery holder, wherein the actuating member can have the design shown or another suitable design depending on the type and design of the manipulating mechanism. The manipulating mechanism includes a hand grip having an axle being rotatably journalled in a tubular housing of the manipulating mechanism and being rigidly connected to the supporting body or the extended rear portion of the artery holder, wherein the axle supports a knob or the like for manual turning of the entire artery holder in relation to the hand grip, e.g. 180°, for enabling the correct positioning of the clamp elements in relation to the cut artery and subsequently, after gripping the artery via its enclosing muscle tissue, for fixing the artery in relation to the opened coronary vessel. Furthermore, the artery holder can be provided with a joint, e.g. just behind the support 25, or be pivotally connected to said axle via a joint, wherein the axle is tubular and contains pull strips being connected to the artery holder and capable of being pulled to-and-fro by means of a manually actuated pulling mechanism being arranged in the hand grip. By means of such a device, the artery holder can be inclined in a plane making an angle with said axle, e.g. 45°, in both directions in relation to a straight starting position. Furthermore, the manipulating mechanism includes a suitable device for actuating the actuating member of the artery holder in order to open and close the clamp elements.

1. An artery holder for use in connection with heart surgery, characterized in that it comprises
a gripping and fixing member (1) including two mutually interacting first and second clamp elements (9, 10) being arranged for gripping muscle tissue (4), that encloses an artery (5), in connection with a cut end (7) of the artery (5), and for clamping muscle tissue (4) while simultaneously fixing the artery (5) and exposing said cut end (7),
a spring member (2) being arranged to actuate the gripping and fixing member (1) with a predetermined spring force in order to achieve said clamping of the muscle tissue (4), and
an actuating member (3) being arranged to actuate the gripping and fixing member (1) against the action of the spring force from the spring member (2) in order to open the clamp elements (9, 10) for enabling reception of muscle tissue (4) therebetween and subsequent release of clamped muscle tissue (4).

2. An artery holder according to claim 1, characterized in that the first clamp element (9), being intended to be located closest to the cut artery end (7), has a central recess (15) at its end edge (11), said recess being open forwards and being defined by two tongues (16, 17) projecting forwards, and being arranged for receiving a portion of the artery (5) when inclining the artery holder in relation to the artery (5), whereas the second clamp element (10) has a straight end edge (12) arranged for supporting against the artery (5) during said inclining of the artery holder.

3. An artery holder according to claim 2, characterized in that said recess (15) is V-shaped.

4. An artery holder according to any one of claims 1-3, characterized in that the gripping and fixing member (1), the spring member (2) and the actuating member (3) are formed by first and second co-operating structural elements (20, 21) being connected to each other by means of an attachment member (22) delimiting the gripping and fixing member (1) from the actuating member (3), said first structural element (20) thereby having the first clamp element (9) and said second structural element (21) having the second clamp element (10).

5. Am artery holder according to claim 4, characterized in that the first structural element (20) consists of a bent and curved spring plate, including an arc-shaped portion forming as well said actuating member (3) as said spring member (2).
